(19) United States
(12) Patent Application Publication
(10) Pub. No.: US 2020/0281944 A1
(43) Pub. Date: Sep. 10, 2020

(54) Combinations Of Lanosterol Or 25-Hydroxycholesterol Including Derivatives Thereof Useful In The Treatment Of Lens Disorders

(71) Applicant: Mahmood Piraee, Montreal (CA)

(72) Inventor: Mahmood Piraee, Montreal (CA)

(21) Appl. No.: 16/764,088

(22) PCT Filed: Nov. 15, 2018
(86) PCT No.: PCT/IB2018/058979

§ 371 Date: May 14, 2020

Related U.S. Application Data
(60) Provisional application No. 62/587,558, filed on Nov. 17, 2017.

(51) Int. Cl. A61K 31/58; C07J 41/00; C07J 43/00; C07J 33/00; C07J 51/00; C07J 31/00; C07J 9/00; A61K 31/575; A61K 31/16; A61K 31/198; A61K 31/4164

(52) U.S. Cl. CPC A61K 31/58; C07J 41/0055; C07J 43/003; C07J 33/002; C07J 51/00; A61K 31/4164; C07J 9/005; A61K 31/575; A61K 31/16; A61K 31/198; C07J 31/006

(57) ABSTRACT

Novel lanosterol derivatives and novel 25-hydroxycholesterol derivatives including their pharmaceutically acceptable salts as well as methods of treatment and pharmaceutical compositions and formulations of lanosterol and derivatives thereof and 25-hydroxycholesterol and derivatives thereof useful in treating ophthalmic disorders including cataracts and presbyopia.

### CROSS-REFERENCE TO RELATED APPLICATIONS

The present application claims the benefit of priority to U.S. Provisional Patent Application No. 62/587,558, filed Nov. 17, 2017, which is incorporated herein by reference in its entirety.

### FIELD

The present disclosure relates to novel lanosterol derivatives and novel 25-hydroxycholesterol derivatives including their pharmaceutically acceptable salts. The present disclosure also relates to compositions of lanosterol or derivatives thereof and 25-hydroxycholesterol or derivatives thereof including combinations with other agents including oxidative protective agents, free radical scavengers, modulators of protein carbonylation, lipid peroxidation, redox or antioxidant enzyme enhancement and modulators of functional telomere length agents; methods of treating cataracts and to kits comprising such compositions.

### BACKGROUND

Cataract is a leading cause of vision impairment, and millions of human patients every year undergo cataract surgery to remove the opacified lenses. Although cataracts can be successfully removed with surgery, this approach is expensive, and most individuals blinded by severe cataracts in developing countries go untreated.

The cost of cataract surgery in the United States estimated at $6 billion each year. Cataract prevalence is increasing and age of onset is decreasing in the developing world. In the next 20 years, as the population ages around the world, cataract surgery is predicted to double. Furthermore, there are no pharmacological treatments to prevent cataract or delay its onset.

Although cataract surgery is the only currently available option for cataract, recent studies have proposed that the transparency of the eye lens depends on maintaining the native tertiary structures and solubility of the lens crystallin proteins. Crystallins are the major component of fiber cells, which form the eyes' lenses, and the unique properties of these cells make them particularly susceptible to damage. In order for our lenses to function well, the crystallins must maintain both the transparency of fiber cells and their flexibility.

Genetic studies have also identified mutations that impair the function of lanosterol synthase (LSS), an enzyme that synthesizes the lens molecule lanosterol which is an amphipathic triterpenoid intermediate compound in the biosynthesis of cholesterol. These studies have demonstrated that lanosterol is important for lens homeostasis and that lack of lanosterol leads to lens opacification, leading to cataract and that lanosterol supplementation has a positive effect on reducing cataract development, and more importantly treating cataract by dissolving the protein aggregates in the lens. (see Zhao et al, “Lanosterol Reverses Protein Aggregation In Cataracts,” Nature. 2015 Jul. 22, DOI: 10.1038, Nature 14650 and J. Fielding Hejtmancik, Ophthalmology, ‘Cataracts Dissolved,” Nature (2015), DOI:10.1038, Nature 14629)

Animals such as dogs can also develop cataracts, and therefore an effective eyedrop treatment could potentially also benefit about 70 million affected pet dogs in the United States.

Presbyopia is an age-related far-sightedness eye disorder that commonly manifests between the ages of 40 and 50, initially causing blurred vision, difficulty seeing in dim light, and eye strain. Presbyopia causes diminishing of vision-targeted life quality and occupational performance for most people over 40 years old. It affects the ability to perform visual tasks at near distance such as book reading, handcrafts, stitching, cooking and surgical operation. Although there are some treatment options such as surgery, the use of near-glasses and contact lenses for presbyopia, topical drug treatment for pharmacological control of presbyopia is currently a very popular and attractive nonsurgical option. It is estimated that there are about 1.8 billion people globally with presbyopia. Persbyopia affects approximately 80% of people over the age of 45. Uncorrected presbyopia causes widespread, avoidable vision impairment throughout the world. There is significant need for innovative, effective and safe treatment options for people with presbyopia. The most common option for treating presbyopia is the use of near-glasses and contact lenses for presbyopia. However topical drug treatment for pharmacological control of presbyopia is currently a very popular option. Current treatment modalities for Presbyopia are based on treatments for reducing pupil sizes by muscarinic agonistic agents such as pilocarpine and physostigmine. Lipoic acid choline ester 1.5% is another compound that is tested successfully for treatment of presbyopia. The compounds which is called EVO06 improved binocular near acuity. EV06 is a prodrug that after penetration into the cornea gets hydrolyzed by esterases to two natural substances: lipoic acid and choline. Enzymes within the lens fiber cells chemically reduce lipoic acid to the active form dihydrolipoic acid. Administration of EV06 increases lens elasticity by decreasing the number of protein-disulfide bonds and makes the crystalline lens more elastic and softer via natural un-crosslinking. EV06 demonstrated improvement in all distance corrected near vision acuity (DCNVA) efficacy measures. (Burns B, Encore Vision Reports Positive Phase I/II Results, 2016. Available at: http://bit.ly/EV06presbyopia (accessed 14 Oct. 2017). Ophthalmology. 2018 October;125(10):1492-1499. doi: 10.1016/j.ophtha.2018.04.013. Global Prevalence of Presbyopia and Vision Impairment from Uncorrected Presbyopia: Systematic Review, Meta-analysis, and Modelling. Fricke TR, Tahhan N, Resnikoff S, Papas E, Burnett A, Ho SM, Naduvilath T, Naidoo KS

### SUMMARY

Lanosterol derivatives and 25-hydroxycholesterol derivatives such as esters have increased potency in reducing cataract formation and dissolving the protein aggregates in the eye lens of patients (e.g., a mammal including human patients and animal patients including dogs) suffering from lens opacity disorders including infant cataracts, and lanosterol or derivatives thereof or 25-hydroxycholesterol or derivatives thereof used in combination with oxidative protective agents such N-acetylcysteine (NAC), or free radical scavenger such as N-acetylcysteine amide (NACA), or a modulator of functional telomere length such as N-acetylcarnosine have a synergistic result in reducing cataract development and can actually reverse cataract development, leading to increased lens clarity and visual acuity, in some cases.

Lanosterol and derivatives thereof as well as 25-hydroxycholesterol and derivatives thereof, such as ester derivatives may also be useful in treating presbyopia because they may increase lens elasticity by decreasing the number of protein-disulfide bonds and make the crystalline lens more elastic and softer via natural un-crosslinking.

Lanosterol and derivatives thereof have the Formula

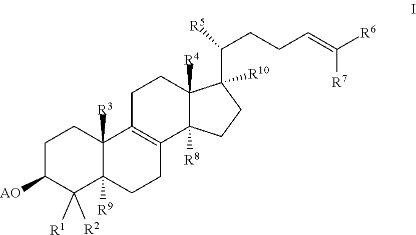

wherein R¹ through R⁸ are each independently selected from hydrogen or lower alkyl optionally substituted by one to three fluoro (in lanosterol each of R¹ through R⁸ are methyl);

R⁹ and R¹⁰ are each independently selected from hydrogen and fluoro (in lanosterol each of R⁹ and R¹⁰ are hydrogen); and

A is H or a hydroxyl derivative such as an ester, thioester, ether, carbamyl, carbonyl, phosphoro functionality (in lanosterol A is hydrogen), and pharmaceutically acceptable salts thereof. Hydroxyl derivatives esterified to lanosterol at A may include a hydrocarbon selected from C₁-C₁₅ alkyl, C₂-C₁₅ alkenyl and C₂-C₁₅ alkynyl each with at least one of an ester functionality, thioester functionality, ether functionality, carbamyl functionality, carbonyl functionality and phosphoro functionality wherein the functionality is esterified to Formula I at A and the hydrocarbon is optionally substituted with at least one of a group selected from —SH, —S—, —NH₂, a guanidine group, a heterocycle, an amide group, —COOH, an epoxide group, and a heterocycle. Preferred hydroxy derivatives esterified to lanosterol at A include N-acetylcysteine (NAC) and alpha-lipoic acid.

25-Hydroxycholesterol and its derivatives have the Formula

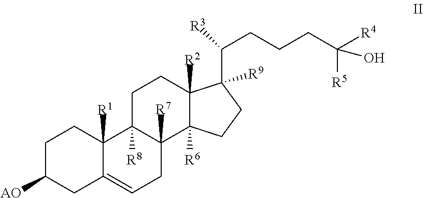

wherein R¹ through R⁸ are each independently selected from hydrogen or lower alkyl optionally substituted by one to three fluoro (in 25-hydroxycholesterol each of R¹ through R⁵ are methyl and R⁶ through R⁸ are hydrogen);

R⁹ is selected from hydrogen and fluoro (in 25-hydroxycholesterol R⁹ is hydrogen); and

A is H or a hydroxyl derivative such as an ester, thioester, ether, carbamyl, carbonyl, phosphoro functionality (in 25-hydroxycholesterol A is hydrogen), and pharmaceutically acceptable salts thereof. Hydroxyl derivatives esterified to 25-hydroxycholesterol at A include N-acetylcysteine (NAC) and alpha-lipoic acid.

In another embodiment, the aspects of the present disclosure are directed to a method of treating an eye cataract in a mammal, including a human being, in need of such treatment comprising administering to said mammal a therapeutically effective amount of a lanosterol derivative.

In another embodiment, the aspects of the present disclosure are directed to a method of treating an eye cataract in a mammal, including a human being, in need of such treatment comprising administering to said mammal a therapeutically effective amount of a 25-hydroxycholesterol derivative.

In another embodiment, the aspects of the present disclosure are directed to a method of stabilizing proteins in an eye of a mammal, including a human being, in need of such treatment comprising administering to said mammal a therapeutically effective amount of lanosterol or a derivative thereof.

In another embodiment, the aspects of the present disclosure are directed to a method of stabilizing proteins in an eye of a mammal, including a human being, in need of such treatment comprising administering to said mammal a therapeutically effective amount of 25-hydroxycholesterol or a derivative thereof.

In another embodiment, the aspects of the present disclosure are directed to a method of treating an eye cataract in a mammal, including a human being, in need of such treatment comprising administering an effective amount of lanosterol or a derivative thereof and an oxidative protective agent. In another embodiment, the aspects of the present disclosure are directed to a method wherein the oxidative protective agent is N-acetylcysteine (NAC) or N-acetylcysteine amide (NACA).

In another embodiment, the aspects of the present disclosure are directed to a method of treating an eye cataract in a mammal, including a human being, in need of such treatment comprising administering an effective amount of 25-hydroxycholesterol or a derivative thereof and an oxidative protective agent. In another embodiment, the aspects of the present disclosure are directed to a method wherein the oxidative protective agent is N-acetylcysteine (NAC) or N-acetylcysteine amide (NACA).

In another embodiment, the aspects of the present disclosure are directed to a method of treating an eye cataract in a mammal, including a human being, in need of such treatment comprising administering an effective amount of lanosterol or a derivative thereof and a free radical scavenger, modulator of protein carbonylation, oxidative protective agent, or a lipid peroxidation, redox, or antioxidant enzyme enhancement. In another embodiment, the aspects of the present disclosure are directed to a method wherein the free radical scavenger, modulator of protein carbonylation, or lipid peroxidation, redox, or antioxidant enzyme enhancement is N-acetylcysteine (NAC) or N-acetylcysteine amide (NACA)).

In another embodiment, the aspects of the present disclosure are directed to a method of treating an eye cataract in a mammal, including a human being, in need of such treatment comprising administering an effective amount of 25-hydroxycholesterol or a derivative thereof and a free radical scavenger, modulator of protein carbonylation, oxidative protective agent, or a lipid peroxidation, redox, or antioxidant enzyme enhancement. In another embodiment, the aspects of the present disclosure are directed to a method wherein the free radical scavenger, modulator of protein carbonylation, or lipid peroxidation, redox, or antioxidant enzyme enhancement is N-acetylcysteine (NAC) or N-acetylcysteine amide (NACA)).

In another embodiment, the aspects of the present disclosure are directed to a method of treating an eye cataract in a mammal, including a human being, in need of such treatment comprising administering an effective amount of lanosterol or a derivative thereof and a modulator of functional telomere length. In another embodiment, the aspects of the present disclosure are directed to a method wherein the modulator of functional telomere length is N-acetylcarnosine.

In another embodiment, the aspects of the present disclosure are directed to a method of treating an eye cataract in a mammal, including a human being, in need of such treatment comprising administering an effective amount of 25-hydroxycholesterol or a derivative thereof and a modulator of functional telomere length. In another embodiment, the aspects of the present disclosure are directed to a method wherein the modulator of functional telomere length is N-acetylcarnosine.

In another embodiment, the aspects of the present disclosure are directed to a method of treating an eye cataract in a mammal, including a human being, in need of such treatment comprising administering an effective amount of lanosterol or a derivative thereof and at least one compound selected from N-acetylcysteine (NAC), N-acetylcysteine amide (NACA) and N-acetylcarnosine.

In another embodiment, the aspects of the present disclosure are directed to a method of treating an eye cataract in a mammal, including a human being, in need of such treatment comprising administering an effective amount of 25-hydroxycholesterol or a derivative thereof and at least one compound selected from N-acetylcysteine (NAC), N-acetylcysteine amide (NACA) and N-acetylcarnosine.

In another embodiment, the aspects of the present disclosure are directed to an ophthalmic composition comprising lanosterol or a derivative thereof and at least one compound selected from N-acetylcysteine (NAC), N-acetylcysteine amide (NACA) and N-acetylcarnosine in a physiologically acceptable buffer, having a pH of 5.0 to 8.0, wherein the lanosterol or a derivative thereof are present at a concentration ranging from about 0.010% w/v to about 5% w/v and each of said compounds N-acetylcysteine (NAC), N-acetylcysteine amide (NACA) or N-acetylcarnosine is present at a concentration ranging from about 0.01% w/v to about 2.00% w/v. In another embodiment, the aspects of the present disclosure are directed to an ophthalmic composition wherein the composition is a topical formulation. In another embodiment, the aspects of the present disclosure are directed to an ophthalmic composition wherein the composition is a formulation, such as, for example, an aqueous formulation or an emulsion formulation such as an oil-containing emulsion. In another embodiment, the aspects of the present disclosure are directed to an aqueous ophthalmic composition wherein the concentration of the lanosterol or a derivative thereof is present in an amount from about 0.10% w/v to about 2.00% w/v. In another embodiment, the aspects of the present disclosure are directed to an aqueous ophthalmic composition wherein the concentration of N-acetylcysteine (NAC), N-acetylcysteine amide (NACA) or N-acetylcarnosine is present in an amount from about 0.01% w/v to 1.2% w/v. In another embodiment, the aspects of the present disclosure are directed to an aqueous ophthalmic composition wherein the pH is in a range from about 5.5 to about 7.0.

In another embodiment, the aspects of the present disclosure are directed to an ophthalmic composition comprising 25-hydroxycholesterol or a derivative thereof and at least one compound selected from N-acetylcysteine (NAC), N-acetylcysteine amide (NACA) and N-acetylcarnosine in a physiologically acceptable buffer, having a pH of 5.0 to 8.0, wherein the 25-hydroxycholesterol or a derivative thereof are present at a concentration ranging from about 0.010% w/v to about 5% w/v and each of said compounds N-acetylcysteine (NAC), N-acetylcysteine amide (NACA) or N-acetylcarnosine is present at a concentration ranging from about 0.01% w/v to about 2.00% w/v. In another embodiment, the aspects of the present disclosure are directed to an ophthalmic composition wherein the composition is a topical formulation. In another embodiment, the aspects of the present disclosure are directed to an ophthalmic composition wherein the composition is an aqueous formulation. In another embodiment, the aspects of the present disclosure are directed to an aqueous ophthalmic composition wherein the concentration of 25-hyroxycholesterol or a derivative thereof is present in an amount from about 0.10% w/v to about 2.00% w/v. In another embodiment, the aspects of the present disclosure are directed to an aqueous ophthalmic composition wherein the concentration of N-acetylcysteine (NAC), N-acetylcysteine amide (NACA) or N-acetylcarnosine is present in an amount from about 0.01% w/v to 1.2% w/v. In another embodiment, the aspects of the present disclosure are directed to an aqueous ophthalmic composition wherein the pH is in a range from about 5.5 to about 7.0.

In another embodiment, the aspects of the present disclosure are directed to a pharmaceutical composition further comprising pharmaceutically acceptable excipients including stabilizers, penetration enhancers, surfactants, polymer base carriers like gelling agents, organic co-solvents, pH active components, osmotic active components and preservatives.

In another embodiment, the aspects of the present disclosure are directed to a kit including a unit dose of an aqueous ophthalmic solution comprising lanosterol or a derivative thereof and at least one compound selected from N-acetylcysteine (NAC), N-acetylcysteine amide (NACA) or N-acetylcarnosine in a physiologically acceptable buffer, having a pH of 5.0 to 8.0, wherein the lanosterol or a derivative thereof are present at a concentration ranging from about 0.01% w/v to about 2.00% w/v and said compound N-acetylcysteine (NAC), N-acetylcysteine amide (NACA) or N-acetylcarnosine is present at a concentration ranging from about 0.01% w/v to about 1.00% w/v, wherein the unit dose is contained within a vial prepared from a pharmaceutically acceptable packaging material. In another embodiment, the aspects of the present disclosure are directed to a kit wherein the unit dose is about 50 μL.

In another embodiment, the aspects of the present disclosure are directed to a kit including a unit dose of an ophthalmic solution comprising 25-hydroxycholesterol or a derivative thereof and at least one compound selected from N-acetylcysteine (NAC), N-acetylcysteine amide (NACA) or N-acetylcarnosine in a physiologically acceptable buffer, having a pH of 5.0 to 8.0, wherein the 25-hydroxycholesterol or a derivative thereof are present at a concentration ranging from about 0.01% w/v to about 2.00% w/v and said compound N-acetylcysteine (NAC), N-acetylcysteine amide (NACA) or N-acetylcarnosine is present at a concentration ranging from about 0.01% w/v to about 1.00% w/v, wherein the unit dose in contained within a vial prepared from a pharmaceutically acceptable packaging material. In another embodiment, the aspects of the present disclosure are directed to a kit wherein the unit dose is about 50 μL..

### DETAILED DESCRIPTION

The present disclosure is generally directed towards lanosterol or derivatives thereof and 25-hydroxycholesterol or derivatives thereof in combination with other active agents and methods for the treatment of eye lens disorders. As will be understood, the various scenarios described herein are only examples, and there are many other scenarios to which the present disclosure will apply.

The compounds of the present disclosure include compounds of Formula I and compounds of Formula II as hereinbefore defined, including all polymorphs and crystal habits thereof, prodrugs and isomers thereof (including optical, geometric and tautomeric isomers) as hereinafter defined and isotopically-labeled compounds of Formula I and Formula II.

“Heterocycle” refers to a saturated, unsaturated or aromatic ring comprising carbon atoms and one or more heteroatoms. Exemplary heteroatoms include N, O, Si, P, B, and S atoms. Heterocycle may be monocyclic or polycyclic and may include 3- to 10-membered monocyclic rings.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl).

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl).

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl).

“Lower alkyl” refers to alkyl groups comprising from one to eight carbon atoms.

The “A” group functionalization uses methods well known to those skilled in the art.

Esterification of free hydroxyl group of lanosterol or 25-hydroxycholesterol (wherein A is hydrogen) includes carboxylic groups such as those of the following prodrug/derivative forming compounds:
1. L-Arginine; L-Arginine is an attractive compound for the potential delay of senile cataracts for multiple reasons. L-Arginine is the single major damaged crystallin residue by advanced glycation during aging. L-Arginine could have stabilizing effects on protein solutions by preventing aggregation and improving solubilization. Local delivery of L-arginine to the eye has low toxicity since it is a natural constituent. Topical application of L-arginine blocks advanced glycation by ascorbic acid in the lens of hSVCT2 transgenic mice. Xingjun Fan, Liu Xiaoqin, Breshey Potts, Christopher M. Strauch, Ina Nemet, Vincent M. Monnier Mol Vis. 2011; 17: 2221-2227.
2. Carnosine; carnosine helps inhibit fibrillation (formation of alpha-crystallin fibrils) in the lens. The addition of carnosine to pre-existing fibrils also could help dissolve the fibrils. L-carnosine prevents lens opacification by 50 to 60 percent in cell cultures in the presence of guanidine, which causes lens opacification. Protective effects of L- and D-carnosine on alpha-crystallin amyloid fibril formation: implications for cataract disease. Attanasio F, Cataldo S, Fisichella S, Nicoletti S, Nicoletti VG, Pignataro B, Savarino A, Rizzarelli E. Biochemistry. 2009 Jul. 14; 48(27):6522-31. doi: 10.1021/bi900343n. Natural dipeptides as mini-chaperones: molecular mechanism of inhibition of lens βL-crystallin aggregation. Dizhevskaya A K, Muranov K O, Boldyrev A A, Ostrovsky M A. Curr Aging Sci. 2012 December; 5(3):236-41. Analytical and physicochemical characterization of the senile cataract drug dipeptide β-alanyl-L-histidine (carnosine).Abdelkader H, Swinden J, Pierscionek B K, Alany R G. J Pharm Biomed Anal. 2015 Oct. 10; 114:241-6. doi: 10.1016/j.jpba.2015.05.025.
3. A phosphoester of S-(3-amino-2-hydroxypropyl)phosphorothioate;
4. Ursodeoxycholic acid (UDCA) and tauroursodeoxycholic acid (TUDCA), It is believed that these two naturally occurring intermediate waste products in the lens enhance the chaperone activity of α-crystallin. These bile acids may protect α-crystallin from aggregation or protect the chaperone activity of α-crystallin. Cholesterol-derived bile acids enhance the chaperone activity of α-crystallins Shuhua Song, Jack J. N. Liang, Michael L. Mulhern, Christian J. Madson, Toshimichi Shinohara Cell Stress Chaperones. 2011 September; 16(5): 475-480. Published online 2011 Mar 6. doi: 10.1007/s12192-011-0259-5. Ursodeoxycholic acid prevents selenite-induced oxidative stress and alleviates cataract formation: In vitro and in vivo studies Hui-Ping Qi, Shu-Qin Wei, Xiang-Chun Gao, Nan-Nan Yu, Wan-Zhen Hu, Sheng Bi, Hao Cui Mol Vis. 2012; 18: 151-160.
5. Alpha-lipoic acid; may possess protective effect on the lens by inducing major biochemical changes in the lens such as increasing glutathione, ascorbate, and vitamin E levels. Alpha-lipoic acid is involved in direct protection of lens protein thiols. Alpha-lipoic acid prevents buthionine sulfoximine-induced cataract formation in newborn rats. Maitra I, Serbinova E, Trischler H, Packer L.Free Radic Biol Med. 1995 April; 18(4):823-9. Alpha-lipoic acid prevents buthionine sulfoximine-induced cataract formation in newborn rats. Maitra I, Serbinova E, Trischler H, Packer L. Free Radic Biol Med. 1995 Apr;18(4):823-9. Effects of two antioxidants; α-lipoic acid and fisetin against diabetic cataract in mice. Kan E, Kiliçkan E, Ayar A, Çolak R. Int Ophthalmol. 2015 Feb; 35(1):115-20. doi: 10.1007/s10792-014-0029-3.
6. Glutathione esters made via esterification either with the glycyl end or with the glutamyl end;
7. (+)-(2S,3S)-3[(S)-3-methyl-1-(3- methyl butylcarbamoyl)butylcarbamoyl]-2-oxiranecarboxylic acid;
8. Glycine; Ester formation with amino acid glycine could increase water solubility, and create amphiphilic analog suitable for ocular drug delivery. Novel strategies for anterior segment ocular drug delivery. Cholkar K, Patel S P, Vadlapudi A D, Mitra A K. J Ocul Pharmacol Ther. 2013 Mar; 29(2):106-23. doi: 10.1089/jop.2012.0200.
9. Acetyl;
10. tert-Pentanoic acid; and
11. N-acetylcysteine (NAC). NAC has shown potential role in protecting lens against cataracts induced by high oxygen levels, or for preventing post-vitrectomy cataracts, or inhibiting the progression of diabetic cataract at the earlier stage. Wang P, Liu XC, Yan H, Li MY. Hyperoxia-induced lens damage in rabbit: protective effects of N-acetlcysteine. Mol Vis. 2009 Dec. 31; 15:2945-52. Liu XC, Wang P, Yan H. A rabbit model to study biochemical damage to the lens after vitrectomy: effects of N-acetylcysteine. Exp Eye Res. 2009.June; 88(6):1165-70. doi: 10.1016/j.exer.2009.01.001. Zhang S, Chai F Y, Yan H, Guo Y, Harding J J. Effects of N-acetylcysteine and glutathione ethyl ester drops on streptozotocin-induced diabetic cataract in rats. Mol Vis. 2008 May 12; 14:862-70.

Ether analogues of Formula I and Formula II include methyl, ethyl, propyl, butyl, isopropyl, cyclopropyl.

Examples of other important prodrugs/derivatives of Formula I and Formula II include:
1. Phosphoric acid monoester modulates dissolution rate-limited absorption and thus can enhance drug solubility. For example, the “A” group functionalization of lanosterol by a phosphate can increase water solubility with excellent stability.

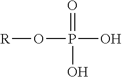

2. Sulfate derivative formation of lanosterol (wherein A is hydrogen) also increases water solubility.
3. Ester formation of lanosterol (wherein A is hydrogen) with N-acetylcysteine (NAC) is a specific embodiment envisioned.
4. Ester formation of 25-hydroxycholesterol (wherein A is hydrogen) with N-acetylcysteine (NAC) is a specific embodiment envisioned.
   Specific prodrug/derivative compounds of formula I have molecular structures such as:

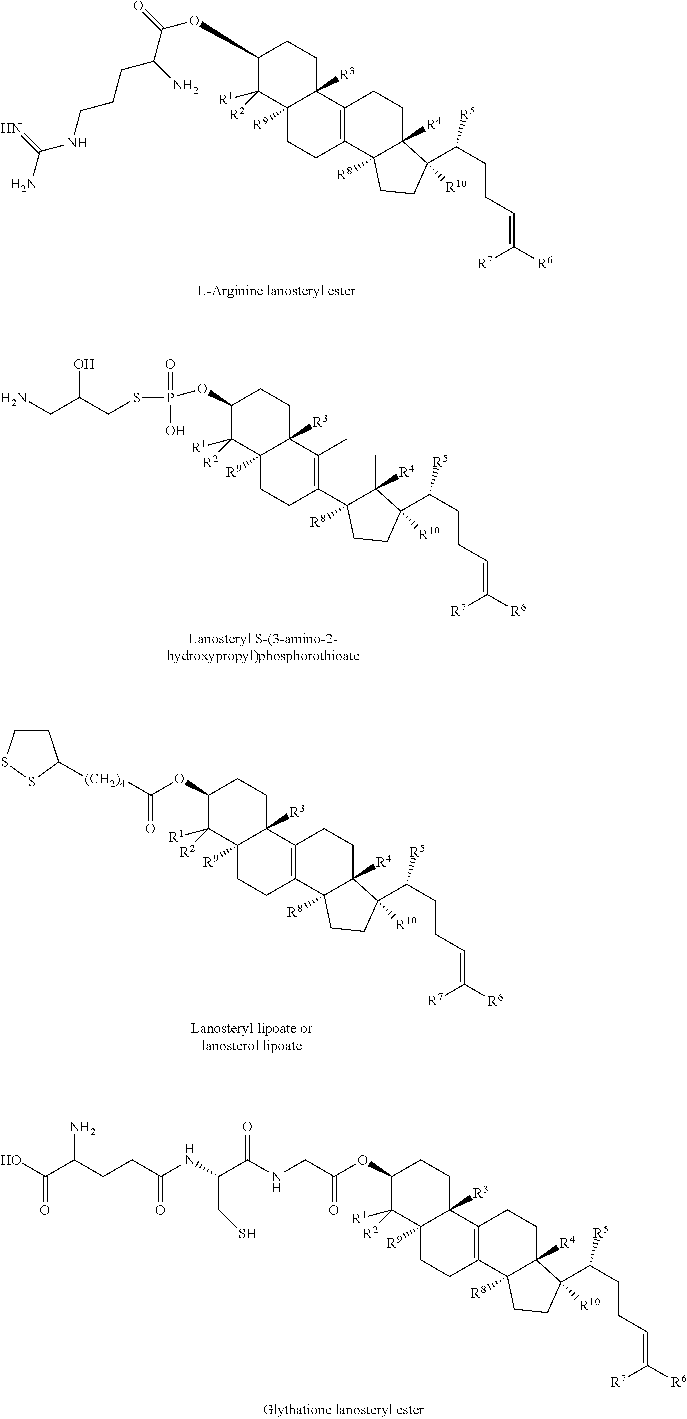

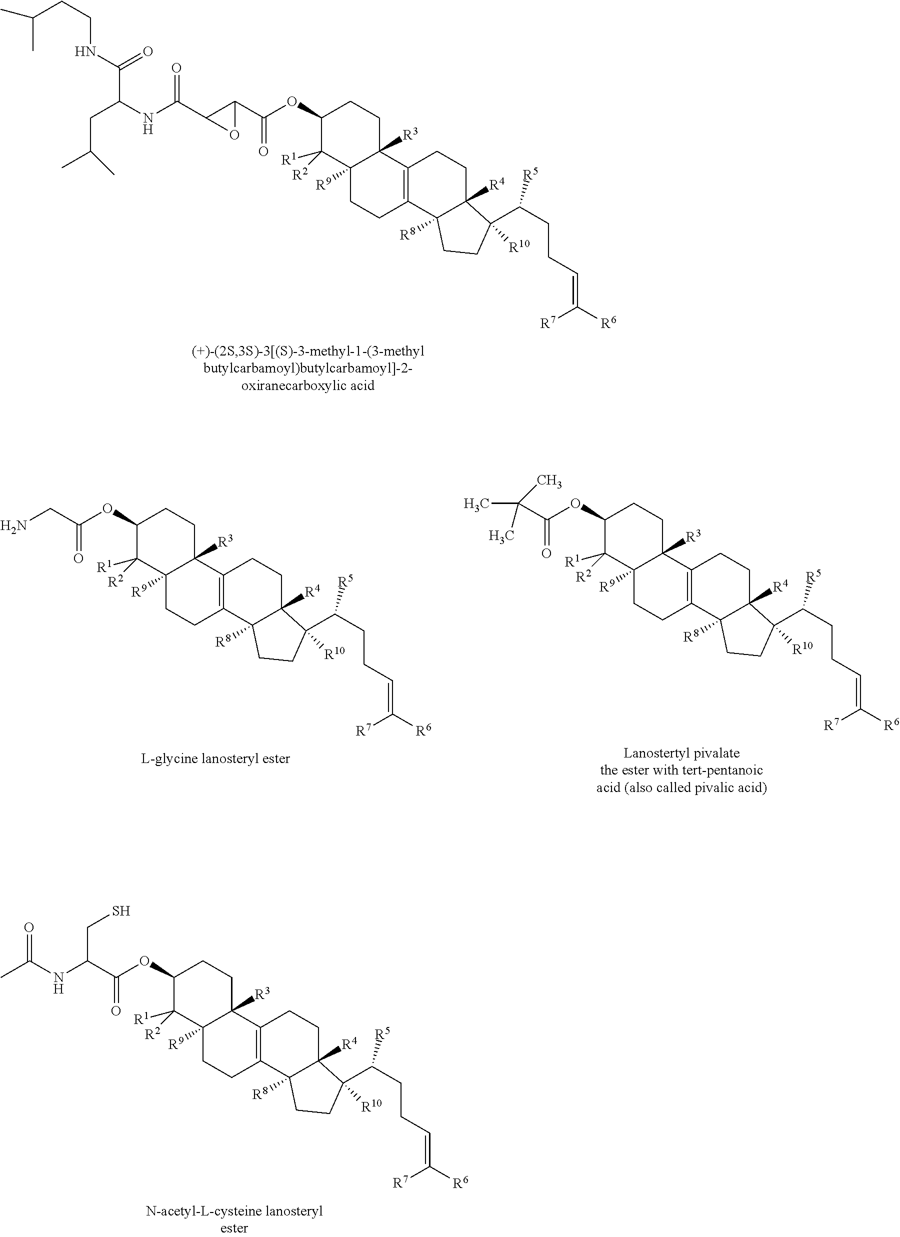

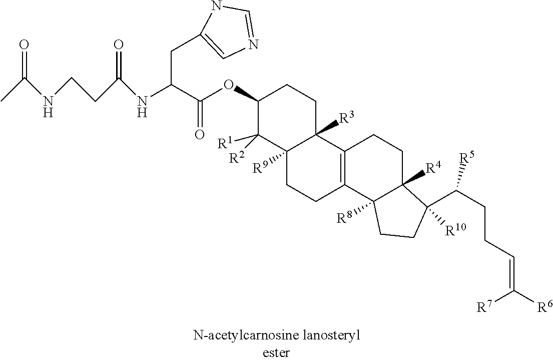

Specific prodrug/derivatives compounds of formula II have molecular structures such as:

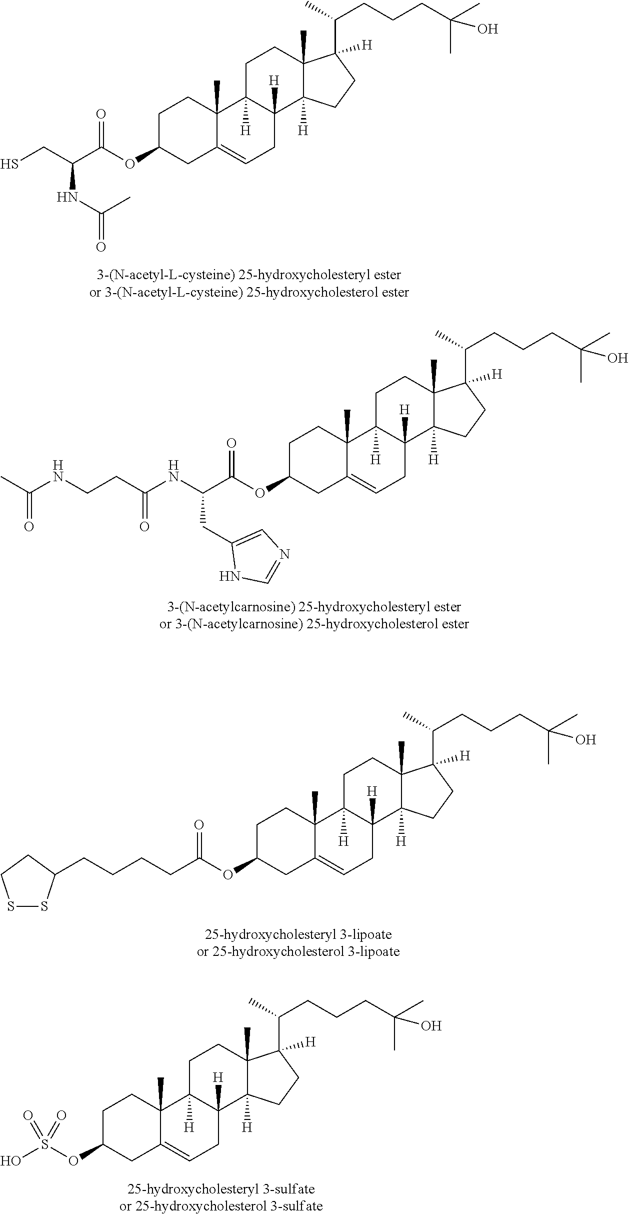

Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association) and Design of Prodrugs by H. Bundgaard (Elsevier, 1985).

Lanosterol is a core steroid and it as well as derivatives thereof can be prepared by numerous methods. Recent data for Lanosterol by Zhao et al, Lanosterol reverses protein aggregation in cataracts. Nature. 2015 Jul. 22. doi: 10.1038/nature14650 and - J. Fielding Hejtmancik. Ophthalmology: Cataracts dissolved. Nature (2015), doi:10.1038/nature14629 has rekindled interest in this compound.

25-Hydroxycholesterol is a core steroid and it as well as derivatives thereof can be prepared by numerous methods. Cholesterol and hydroxycholesterol compounds including compounds, species, compound formula substituent side chains, compositions, formulations, methods of testing and methods of use thereof in the treatment and prevention of ocular conditions including cataract and presbyopia are disclosed in U.S. Pat. Publication No. 2018/0250313, Makley, et al., entitled “Compounds and formulations for treating ophthalmic diseases” the disclosure of which is hereby incorporated by reference in its entirety.

N-acetylcysteine (NAC) is an N-acetylated amino acid required by our bodies to produce glutathione and possesses antioxidant properties capable of reducing inflammatory and catabolic molecules. Inflammatory mechanisms cause the release of arachidonic acid which generates leukotrienes (LTs) which are mediators of ischemia, epithelial destruction and arterial constriction. LTs are produced by many cell types such as, mast cells, leucocytes, connective tissue cells, macrophages, alveolar cells and vascular smooth muscle cells. In addition to NAC, other derivatives of amino acids cysteine and cystine include: N,N′-acetylcystine (N-DAC) and N-acetyl homocysteine (NAH). NAC, N-DAC, NAH interact with peroxides and LTs, reducing toxic free radicals, interrupt the LT cascade and reduce inflammation and promote healing. When NAC is administered with lanosterol or derivative thereof the results are synergistic.

NAC has shown beneficial effects in protecting lens against cataracts induced by high oxygen levels, or for preventing post-vitrectomy cataracts, or inhibiting the progression of diabetic cataract at the earlier stage. Wang P, Liu XC, Yan H, Li MY. Hyperoxia-induced lens damage in rabbit: protective effects of N-acetylcysteine. Mol Vis. 2009;15:2945-52. Liu XC, Wang P, Yan H. A rabbit model to study biochemical damage to the lens after vitrectomy: effects of N-acetylcysteine. Exp Eye Res. 2009; 88(6):1165-70. doi: 10.1016/j.exer.2009.01.001. Zhang S, Chai FY, Yan H, Guo Y, Harding JJ. Effects of N-acetylcysteine and glutathione ethyl ester drops on streptozotocin-induced diabetic cataract in rats. Mol Vis. 2008;14:862-70.

NAC is also described in Epstein, U.S. Pat. No. 5,306,731, as a method for treating or preventing glaucoma by administering NAC. Repine et al, U.S. Pat. No. 5,596,011, discloses a method for treating macular degeneration with a glutathione enhancing agent, and antioxidant and an anti-inflammatory agent, interferon. Mason et al, U.S. Pat. No. 5,691,380, discloses a composition comprising an organopolysiloxane, NAC and an emulsifier. the disclosures of U.S. Pat. Nos. 5,306,731; 5,596,011; and 5,691,380 are hereby incorporated by reference in their entirety.

N-acetylcysteine amide (NACA), the amide form of N-acetylcysteine (NAC), is a low molecular weight thiol antioxidant and a Cu²⁺ chelator. NACA provides protective effects against cell damage. NACA has been shown to inhibit t-butylhydroxyperoxide (BuOOH)-induced intracellular oxidation in red blood cells (RBCs) and to retard BuOOH-induced thiol depletion and hemoglobin oxidation in the RBCs. This restoration of thiol-depleted RBCs by externally applied NACA was significantly greater than that found using NAC. Unlike NAC, NACA protected hemoglobin from oxidation. (L. Grinberg et al., Free Radic Biol Med., 2005 Jan. 1, 38(1):136-45). In a cell-free system, NACA was shown to react with oxidized glutathione (GSSG) to generate reduced glutathione (GSH). NACA readily permeates cell membranes, replenishes intracellular GSH, and, by incorporating into the cell's redox machinery, protects the cell from oxidation. Because of its neutral carbonyl group, NACA possesses enhanced properties of lipophilicity and cell permeability. (See, e.g., U.S. Pat. No. 5,874,468 to D. Atlas et al.). NACA is also superior to NAC in crossing the cell membrane.

NACA may function directly or indirectly in many important biological phenomena, including the synthesis of proteins and DNA, transport, enzyme activity, metabolism, and protection of cells from free-radical mediated damage. NACA is a potent cellular antioxidant responsible for maintaining the proper oxidation state within the body. NACA can recycle oxidized biomolecules back to their active reduced forms and may be as effective, if not more effective, than GSH as an antioxidant. NACA can inhibit cataract formation by limiting protein carbonylation, lipid peroxidation, and redox system components, as well as replenishing antioxidant enzymes. Carey JW, Pinarci EY, Penugonda S, Karacal H, Ercal N. In vivo inhibition of I-buthionine-(S,R)-sulfoximine-induced cataracts by a novel antioxidant, N-acetylcysteine amide. Free Radic Biol Med. 2011 Mar. 15; 50(6):722-9. doi: 10.1016/j.freeradbiomed.2010.12.017.

When NACA is administered with lanosterol or derivative thereof the results are synergistic.

Free radical scavengers include N-acetylcysteine (NAC) and N-acetylcysteine amide (NACA).

Modulators of protein carbonylation include N-acetylcysteine (NAC) and N-acetylcysteine amide (NACA).

Oxidative protective agents include N-acetylcysteine (NAC) and N-acetylcysteine amide (NACA).

Lipid peroxidation, redox, or antioxidant enzyme enhancers include N-acteylcysteine (NAC) and N-acetylcysteine amide (NACA).

Modulators of functional telomere length include N-acetylcarnosine.

Methods of preparing and using N-acetylcarnosine are described in WO2004028536 published 2004-Apr.-8 and WO9510294 published 1995-Apr.-20. See also http://www.vita-stream.com/can-c-eye-drops.html, which explains a marketed eye drop containing 1% N-acetylcarnosine; also see (http://www.ncbi.nlm.nih.gov/pubmed/24783234). Other N-acetylcarnosine publications include Babizhayev MA,Yegorov YE (2014). Biomarkers of oxidative stress and cataract. Novel drug delivery therapeutic strategies targeting telomere reduction and the expression of telomerase activity in the lens epithelial cells with N-acetylcarnosine acetylcarnosine lubricant eye drops: anti-cataract which helps to prevent and treat cataracts in the eyes of dogs and other animals. N-acetylcarnosine is indicated in therapeutic treatment of cataracts in canines through targeting the prevention of loss of functional telomere length below a critical threshold. Curr Drug Deliv., 2014; 11(1):24-61; Babizhayev M A. Ocular drug metabolism of the bioactivating antioxidant N-acetylcarnosine for vision in ophthalmic prodrug and codrug design and delivery. Drug Dev Ind Pharm., 2008 Oct;34(10):1071-89. doi: 10.1080/03639040801958413; and Babizhayev M A, Burke L, Micans P, Richer S P. N-Acetylcarnosine sustained drug delivery eye drops to control the signs of ageless vision: glare sensitivity, cataract amelioration and quality of vision currently available treatment for the challenging 50,000-patient population. Clin Intery Aging. 2009; 4:31-50. Epub 2009 May 14. When N-acetylcarnosine is administered with lanosterol or derivative thereof the results are synergistic.

Other combination agents include corticosteroids, diuretics, antidiabetic agents, lutein, zeaxanthin, crocin, nitric oxide synthase inhibitors, resveratrol, beta hydroxy acid, N-acetylcysteine, ascorbityl palmitate, ascorbic acid, alpha-lipoic acid, glutathione, methyl-sulfonyl-methane, zinc compounds, aloe vera, antioxidants, vitamins, minerals, and amino acids.

One specific embodiment relates to an ophthalmic formulation of lanosterol or 25-hydroxycholesterol, N-acetylcysteine (NAC), N-acetylcysteine amide (NACA) and N-acetylcarnosine. When, N-acetylcysteine (NAC), N-acetylcysteine amide (NACA) and N-acetylcarnosine are administered with lanosterol or derivatives or with 25-hydroxycholesterol or derivatives thereof, the results are synergistic. As used herein, the term “effective cataract-inhibiting amount” means an amount which will inhibit the progression or formation of cataracts in an eye or inhibit the progression or formation of mature cataracts from developing cataracts already present in the eye. The effective cataract-inhibiting amount of the agent or agents will depend on various factors known to those of ordinary skill in the art. Such factors include, but are not limited to, the size of the eye, the extent and progression of any fully developed or developing cataracts already present in the eye, and the mode of administration. The effective cataract-inhibiting amount will also depend on whether the pharmaceutical composition is to be administered a single time, or whether the pharmaceutical composition is to be administered periodically, over a period of time. The period time may be any number of days, weeks, months, or years. In one embodiment, the effective cataract-inhibiting amount of lanosterol is about 0.010% w/v to about 5% w/v.

Cornea and aqueous humor have significant esterase activity and thus in particular, the so-called ester prodrugs described herein are very important in improving ocular drug delivery. Prodrug derivatization can be further used to enhance drug lipophilicity in order to overcome the permeability barrier. Once the ester prodrug enters the eye the active form of the drug will be released by the function of ocular esterases. For example, NAC and lanosterol are released inside the eye after the hydrolysis of lanosterol NAC ester by ocular esterases.

As used herein the term “ophthalmic composition” refers to a pharmaceutically acceptable formulation, delivery device, mechanism or system suitable for administration to the eye. The term “ophthalmic compositions” includes but are not limited to solutions, suspensions, gels, ointments, sprays, depot devices or any other type of formulation, device or mechanism suitable for short term or long-term delivery of active agent to the eye. Specific ophthalmic compositions are advantageously in the form of ophthalmic solutions or suspensions (i.e., eye drops), ophthalmic ointments, or ophthalmic gels containing active agent. Depending upon the particular form selected, the compositions may contain various additives such as buffering agents, isotonizing agents, solubilizers, preservatives, viscosity-increasing agents, chelating agents, antioxidizing agents, and pH regulators.

The present disclosure also relates to compositions comprising a compound of Formula I or an acceptable salt thereof (e.g., pharmaceutical compositions). Accordingly, in one embodiment, the present disclosure relates to a pharmaceutical composition comprising a compound of Formula I, a pharmaceutically acceptable carrier and, optionally, at least one additional medicinal or pharmaceutical agent.

The disclosure also relates to compositions comprising a compound of Formula II or an acceptable salt thereof (e.g., pharmaceutical compositions). Accordingly, in one embodiment, the disclosure relates to a pharmaceutical composition comprising a compound of Formula II, a pharmaceutically acceptable carrier and, optionally, at least one additional medicinal or pharmaceutical agent.

The pharmaceutically acceptable carrier may comprise any conventional pharmaceutical carrier or excipient. Suitable pharmaceutical carriers include inert diluents or fillers, water and various organic solvents (such as hydrates and solvates). The pharmaceutical compositions may, if desired, contain additional ingredients such as binders, excipients and the like. Examples of the base for eye drops include aqueous solvents such as sterile purified water, physiological saline, and buffer.

Examples of the ingredients for eye ointments preferably include Vaseline, plastibase, liquid paraffin, polyethylene glycol, and carboxymethylcellulose.

The pH of the eye drops of the present disclosure is normally from 3 to 7, preferably from 4 to 6, more preferably from 4.5 to 5.5.

Active compounds may be dissolved or suspended in a suitable solvent.

Additives to be added as appropriate in eye drops are exemplified by the following:

Buffers include phosphate buffer, borate buffer, citrate buffer, tartrate buffer, acetate buffer, and amino acids. Preferred is a buffer having a buffer capacity in the pH range of 2-9.

Isotonizing agents include, for example, sugars such as sorbitol , glucose and mannitol , polyhydric alcohols such as glycerin, polyethylene glycol and propylene glycol and salts such as sodium chloride.

Preservatives include, for example, benzalkonium chloride, benzethonium chloride, p-oxybenzoates such as methyl p-oxybenzoate and ethyl p-oxybenzoate, benzyl alcohol, phenethyl alcohol , sorbic acid and its salt, thimerosal , and chlorobutanol.

Thickeners include, for example, hydroxyethylcellulose, hydroxypropylcellulose, methylcellulose, hydroxypropylmethyl-cellulose, and carboxymethylcellulose and its salt.

Solubilizers (stabilizers) include, for example, polymers such as cyclodextrins and polyvinylpyrrolidone, and surfactants such as Polysorbate 80.

Chelating agents include, for example, disodium edetate, sodium citrate, and condensed sodium phosphate.

Suspending agents include, for example, surfactants such as Polysorbate 80, and polymers such as sodium methylcellulose, hydroxpropylmethylcellulose and methylcellulose.

Eye drops can be prepared by mixing a base such as an aqueous solvent, additives and at least one compound of Formula I in an appropriate order, and adjusting the pH of the mixture to 3-7 by an appropriate step, followed by sterilization.

Eye drops can be prepared by mixing a base such as an aqueous solvent, additives and at least one compound of Formula II in an appropriate order, and adjusting the pH of the mixture to 3-7 by an appropriate step, followed by sterilization.

The methods for sterilization are conventional and include, for example, sterilization by filtration, high pressure steam sterilization and flow steam sterilization. One specific method is sterilization by filtration using a 0.22 μm membrane filter.

An eye drop in which the compound has been dissolved may be prepared by adding additives and at least one active ingredient compound of Formula I to a base in a suitable order, and adjusting pH to 3-7. When a buffer is used, the pH is preferably adjusted to 4-6 after the addition of a buffer having a buffer capacity in the pH range of 2-9.

An eye drop in which the compound has been dissolved may be prepared by adding additives and at least one active ingredient compound of Formula II to a base in a suitable order, and adjusting pH to 3-7. When a buffer is used, the pH is preferably adjusted to 4-6 after the addition of a buffer having a buffer capacity in the pH range of 2-9.

An eye drop in which the compound has been suspended may be prepared by adding additives to a base adjusting its pH to 3-7, subjecting the solution to sterilization, and mixing a compound separately sterilized. When a buffer is used, the pH is preferably adjusted to 4-6 after the addition of a buffer having a buffer capacity in the pH range of 2-9.

The pH adjusting agents to be used here may be conventional ones and include, for example, hydrochloric acid, acetic acid, phosphoric acid, sodium hydroxide, and ammonium hydroxide, with preference given to 1N hydrochloric acid and 1N sodium hydroxide.

When the cataract treating agent of the present disclosure is used in the form of an eye ointment, it may be prepared by mixing at least one compound of Formula I with a base conventionally employed for eye ointments and then following the conventional processes.

When the cataract treating agent of the present disclosure is used in the form of an eye ointment, it may be prepared by mixing at least one compound of Formula II with a base conventionally employed for eye ointments and then following the conventional processes.

While the amount of the compound Formula I, which is to be contained in the cataract-treating formulation of the present disclosure varies depending on the kind of the compound to be selected, it is preferably contained in a proportion of about 0.001-10w/v %, more preferably about 0.01-1 w/v % for eye drops and about 0.001-10 w/w %, more preferably about 0.01-1 w/w % for eye ointments.

While the amount of the compound Formula II, which is to be contained in the cataract-treating formulation of the present disclosure varies depending on the kind of the compound to be selected, it is preferably contained in a proportion of about 0.001-10w/v %, more preferably about 0.01-1 w/v % for eye drops and about 0.001-10 w/w %, more preferably about 0.01-1 w/w % for eye ointments.

The pharmaceutical composition may be in unit dosage forms suitable for single administration of precise dosages.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

Administration of the compounds of Formula I may be affected by any method that enables delivery of the compounds to the site of action.

Administration of the compounds of Formula II may be affected by any method that enables delivery of the compounds to the site of action.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the embodiments of the present disclosure.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the active agent are well-known in the relevant art and would be understood to be encompassed by the skilled artisan once provided the teachings disclosed herein.

As used herein, the term “combination therapy” refers to the administration of a compound of Formula I and/or Formula II together with an at least one additional pharmaceutical or medicinal agent, either sequentially or simultaneously.

The present disclosure includes the use of a combination of a compound as provided in Formula I and/or Formula II and one or more additional pharmaceutically active agent(s). If a combination of active agents is administered, then they may be administered sequentially or simultaneously, in separate dosage forms or combined in a single dosage form. Accordingly, the present disclosure also includes pharmaceutical compositions comprising an amount of: (a) a first agent comprising a compound of Formula (I) or a pharmaceutically acceptable salt of the compound; (b) a second pharmaceutically active agent; and (c) a pharmaceutically acceptable carrier, vehicle or diluent. Accordingly, the present disclosure also includes pharmaceutical compositions comprising an amount of: (a) a first agent comprising a compound of Formula (II) or a pharmaceutically acceptable salt of the compound; (b) a second pharmaceutically active agent; and (c) a pharmaceutically acceptable carrier, vehicle or diluent.

The present disclosure is also directed to pharmaceutical compositions comprising a compound of Formula I or a pharmaceutically acceptable salt thereof, and a pharmaceutically acceptable carrier.

The present disclosure is also directed to pharmaceutical compositions comprising a compound of Formula II or a pharmaceutically acceptable salt thereof, and a pharmaceutically acceptable carrier.

Pharmaceutical compositions suitable for the delivery of compounds of the present disclosure and methods for their preparation will be readily apparent to those skilled in the art. Such compositions and methods for their preparation may be found, for example, in Remington's Pharmaceutical Sciences, 19th Edition (Mack Publishing Company, 1995).

A suitable solution formulation for use in an atomizer using electrohydrodynamics to produce a fine mist may contain from 1 μg to 20 mg of the compound of the present disclosure per actuation and the actuation volume may vary from 1 μl to 100 μl. A typical formulation may comprise a compound of Formula I, propylene glycol, sterile water, ethanol and sodium chloride. Alternative solvents which may be used instead of propylene glycol include glycerol and polyethylene glycol.

A suitable solution formulation for use in an atomizer using electrohydrodynamics to produce a fine mist may contain from 1 μg to 20 mg of the compound of the present disclosure per actuation and the actuation volume may vary from 1μl to 100 μl. A typical formulation may comprise a compound of Formula 11, propylene glycol, sterile water, ethanol and sodium chloride. Alternative solvents which may be used instead of propylene glycol include glycerol and polyethylene glycol.

The compounds of the present disclosure may also be administered directly to the eye, typically in the form of drops of a micronized suspension or solution in isotonic, pH-adjusted, sterile saline. Other formulations suitable for ocular administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

The compounds of the present disclosure may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Administration of the unit dose may be repeated every 4 to 24 hours and continued for as long as needed to achieve the desired effect.

Since the present disclosure has an aspect that relates to the treatment of the disease/conditions described herein with a combination of active ingredients which may be administered separately, the present disclosure also relates to combining separate pharmaceutical compositions in kit form. The kit comprises two separate pharmaceutical compositions: a compound of Formula 1 and/or Formula 11 prodrug/derivative thereof or a salt of such compound or prodrug and a second compound as described above. The kit comprises means for containing the separate compositions such as a container, a divided bottle or a single unit dose vial or container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

In another specific embodiment of the present disclosure, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

All publications, including but not limited to, issued patents, patent applications, and journal articles, cited in this application are each herein incorporated by reference in their entirety.

### EXAMPLE 1

| Lanosterol derivative | 25 mM (equivalent to 1.136 g | |
|---|---|---|
| | of free lanosterol) | |
| (EDTA)2 Na | 0.1 | g |
| Alkyldimethylbenzylammonium chloride | 0.005 | g |
| Ethanol | 18.2 | ml |
| Double distilled water (ophthalmic grade) | qs 100 | ml |

| | |
|---|---|
| pH adjusted to | 5.66 |

### EXAMPLE 2

| Lanosterol derivative | 25 mM (equivalent to 1.136 g | |
|---|---|---|
| | of free lanosterol) | |
| N-acetylcarnosine | 0.5 | g |
| (EDTA)2 Na | 0.1 | g |
| Alkyldimethylbenzylammonium chloride | 0.005 | ml |
| Ethanol | 18.2 | ml |
| Double distilled water (ophthalmic grade) | qs 100 | ml |

| | |
|---|---|
| pH adjusted to | 5.66 |

### EXAMPLE 3

| Lanosterol derivative | 25 mM (equivalent to 1.136 g | |
|---|---|---|
| | of free lanosterol) | |
| N-acetylcysteine | 0.55 | g |
| (EDTA)2 Na | 0.1 | g |
| Alkyldimethylbenzylammonium chloride | 0.005 | ml |
| Ethanol | 18.2 | ml |
| Double distilled water (ophthalmic grade) | qs 100 | ml |

| | |
|---|---|
| pH adjusted to | 5.66 |

### EXAMPLE 4

| Lanosterol derivative | 25 mM (equivalent to 1.136 g | |
|---|---|---|
| | of free lanosterol) | |
| N-acetylcysteine | 0.7 | g |
| Dibasic sodium phosphate | 0.8 | g |
| Monobasic sodium phosphate | 0.15 | g |
| Sodium calcium edetate | 0.005 | ml |
| Alkyldimethylbenzylammonium chloride | 0.005 | g |
| Ethanol | 18.2 | ml |
| Double distilled water (ophthalmic grade) | qs 100 | ml |

| | |
|---|---|
| pH adjusted to | 6.2 |

### EXAMPLE 5

| Lanosterol derivative | 25 mM (equivalent to 1.136 g | |
|---|---|---|
| | of free lanosterol) | |
| N-acetylcysteine amide (NACA) | 0.6 | g |
| (EDTA)2 Na | 0.1 | g |
| Alkyldimethylbenzylammonium chloride | 0.005 | g |
| Ethanol | 18.2 | ml |
| Double distilled water (ophthalmic grade) | qs 100 | ml |

| | |
|---|---|
| pH adjusted to | 5.66 |

### EXAMPLE 6

| Lanosterol derivative | 25 mM (equivalent to 1.136 g | |
|---|---|---|
| | of free lanosterol) | |
| N-acetylcarnosine | 0.45 | g |
| (EDTA)2 Na | 0.1 | g |
| Carboxymethylcellulose sodium | 0.3 | g |
| Glycerine | 1 | g |
| Potassium borate | 0.77 | g |
| Potassium bicarbonate | 0.33 | g |
| Alkyldimethylbenzylammonium chloride | 0.005 | g |
| Ethanol | 18.2 | ml |
| Double distilled water (ophthalmic grade) | qs 100 | ml |

| | |
|---|---|
| pH adjusted to | 6.2 |

### EXAMPLE 7

| | | |
|---|---|---|
| Lanosterol | 1.136 | g |
| N-acetylcysteine | 0.7 | g |
| (EDTA)2 Na | 0.1 | g |
| Carboxymethylcellulose sodium | 0.3 | g |
| Glycerine | 1 | g |
| Potassium borate | 0.77 | g |
| Potassium bicarbonate | 0.33 | g |
| Alkyldimethylbenzylammonium chloride | 0.005 | g |
| Ethanol | 18.2 | ml |
| Double distilled water (ophthalmic grade) | qs 100 | ml |

| | |
|---|---|
| pH adjusted to | 6.2 |

### EXAMPLE 8

| Lanosterol derivative | 25 mM (equivalent to 1.136 g | |
|---|---|---|
| | of free lanosterol) | |
| N-acetylcysteine amide (NACA) | 0.5 | g |
| (EDTA)2 Na | 0.02 | g |
| Boric acid | 1.7 | g |
| Sodium tetraborate | 0.4 | g |
| Benzalkonium chloride | 0.005 | g |
| Ethanol | 18.2 | ml |
| Double distilled water (ophthalmic grade) | qs 100 | ml |

| | |
|---|---|
| pH adjusted to | 6.6 |

### EXAMPLE 9

| | | |
|---|---|---|
| 25-hydroxycholesterol derivative | 25 | mM |
| N-acetylcarnosine | 0.45 | g |
| (EDTA)2 Na | 0.1 | g |
| Carboxymethylcellulose sodium | 0.3 | g |
| Glycerine | 1 | g |
| Potassium borate | 0.77 | g |
| Potassium bicarbonate | 0.33 | g |
| Alkyldimethylbenzylammonium chloride | 0.005 | g |
| Ethanol | 18.2 | ml |
| Double distilled water (ophthalmic grade) | qs 100 | ml |

| | |
|---|---|
| pH adjusted to | 6.2 |

### EXAMPLE 10

A patient diagnosed with cataracts and presenting with lens cloudiness has photographic pre-treatment images taken. Two drops of a liquid from Examples 1-9 (including mixed dosing) is applied three times daily to such patient suffering from cataracts for six weeks. Weekly photographic monitoring shows dramatic improvement of lens cloudiness after 3 weeks with almost complete absence of cloudiness after six weeks.

### EXAMPLE 11

N-Acetylcysteine (NAC) ester of Lanosterol

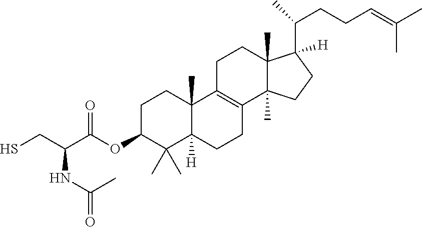 [SMILES: CC(=O)N[C@@H](CS)C(=O)O[C@H]1CC[C@]2(C)C3=C(CC[C@H]2C1(C)C)[C@]1(C)CC[C@H]([C@H](C)CCC=C(C)C)[C@@]1(C)CC3]

N-Acetyl-L-cysteine lanosteryl ester

Synthetic procedure reported for the preparation of amides of N-acetyl-L-cysteine: Uzma I. Zakai, Galina Bikzhanova, Daryl Staveness, Stephen Gately, Robert West. Synthesis of lipophilic sila derivatives of N-acetylcysteineamide, a cell permeating thiol.

Appl. Organometal. Chem. 2010, 24, 189-192.

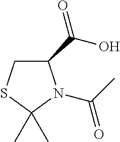

(R)-4-carboxy-3-acetyl-2,2-dimethylthiazolidine was prepared as follows. A suspension of N-acetyl-L-cysteine (1.0 g, 6 mmol) and montmorillonite K10 (0.2 g, 20 wt %) in anhydrous acetone-2,2-dimethoxypropane mixture (1 : 3, 40 mL) is stirred at room temperature for 3 h. The reaction mixture is then filtered, and solvent is evaporated to give (R)-4-carboxy-3-acetyl-2,2-dimethylthiazolidine (1.12 g, 95% yield) as white solid (90% pure) which is further purified by recrystallization from acetone-hexane (1.03 g, 84% yield). ¹H NMR (acetone-d₆, 500 MHz) δ 5 1.48 (s, 3 H, Me), 1.50 (s, 3 H, Me), 1.95 (s, 3 H, NAc), 2.86 (dd, J=13.3, 7.1, 1 H, CHH), 3.00 (dd, J=13.3, 5.1, 1 H, CHH), 4.67 (ddd, J=8.0, 7.1, 5.1, 1 H, CH), 12.80 (br. s, 1 H, OH).

A solution of (R)-4-carboxy-3-acetyl-2,2-dimethylthiazolidine (1 mmol) and triethylamine (1 mmol) in dichloromethane (DCM, 4 mL) is cooled to −5° C., and a solution of ethyl chloroformate (1 mmol) in dichloromethane (1 mL) is added dropwise. After 15 min of stirring at -5° C., lanosterol (1 mmol) is slowly added to the reaction mixture. Stirring is continued for 25 min at −5° C. and 15 h at room temperature. The reaction mixture is then diluted with DCM (6 mL) and washed thoroughly with portions of 5% hydrochloric acid, sodium bicarbonate and water (6 mL each). The dichloromethane solution is dried (MgSO₄) and evaporated to give the dimethylthiazolidine ester intermediate. A solution of the dimethylthiazolidine ester intermediate in 2 M HCI methanolic solution (15 mL) is stirred at room temperature for 24 h. The methanol is removed under reduced pressure and the reaction worked up with DCM-brine. The dichloromethane solution is dried (MgSO₄), filtered and evaporated to give the lanosteryl ester of N-acetyl-L-cysteine.

### EXAMPLE 12

N-Acetylcarnosine ester of Lanosterol

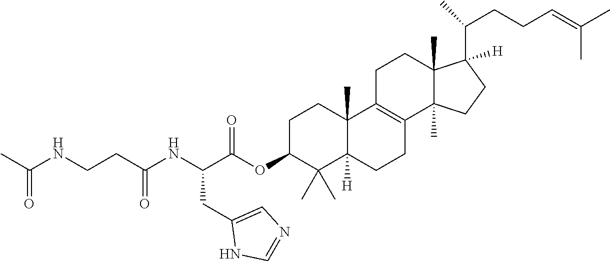 [SMILES: CC(=O)NCCC(=O)N[C@@H](Cc1cnc[nH]1)C(=O)O[C@H]1CC[C@]2(C)C3=C(CC[C@H]2C1(C)C)[C@]1(C)CC[C@H]([C@H](C)CCC=C(C)C)[C@@]1(C)CC3]

N-Acetyl-L-carnosine lanosteryl ester

Synthetic procedure reported for the esterification of N-acetylamino acids: Thonthula Sreelatha, Atmakur Hyavathi, Katragadda Suresh Babu, Joish Madhusudana Murthy, Usha Rani Pathipati, Janaswamy Madhusudana Rao. Synthesis and Insect Antifeedant Activity of Plumbagin Derivatives with the Amino Acid Moiety.

J. Agric. Food Chem. 2009, 57, 6090-6094.

N,N′-Dicyclohexylcarbodimide (DCC, 1.5 mmol) and a catalytic amount of 3-hydroxybenzotriazole are added to a cooled solution (0° C.) of lanosterol (1 mmol) in dry dichloromethane (DCM, 10 mL) under a nitrogen atmosphere. After stirring for 15 min, N-acetyl-L-carnosine (1.2 mmol) is added, and stirring is continued at room temperature. After completion of the reaction (monitored by TLC), the reaction mixture is filtered to remove the precipitated dicyclohexylurea. The filtrate is evaporated under reduced pressure, and the residue is purified by silica gel (100-200 mesh) column chromatography using dichloromethane/methanol as eluent to afford the lanosteryl ester of N-acetyl-L-carnosine.

### EXAMPLE 13

L-Carnosine ester of Lanosterol

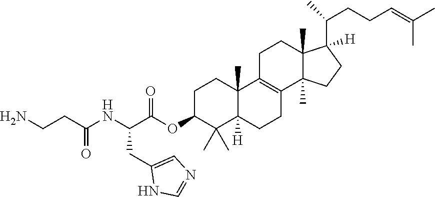 [SMILES: CC(C)=CCC[C@@H](C)[C@H]1CC[C@@]2(C)C3=C(CC[C@]12C)[C@@]1(C)CC[C@H](OC(=O)[C@H](Cc2cnc[nH]2)NC(=O)CCN)C(C)(C)[C@@H]1CC3]

L-Carnosine lanosteryl ester

Reported for the isopropyl ester:

Marica Orioli, Giulio Vistoli, Luca Regazzoni, Alessandro Pedretti, Annunziata Lapolla, Giuseppe Rossoni, Renato Canevotti, Luca Gamberoni, Massimo Previtali, Marina Carini, Giancarlo Aldini.

Design, Synthesis, ADME Properties, and Pharmacological Activities of β-Alanyl-D-histidine (D-Carnosine) Prodrugs with Improved Bioavailability. ChemMedChem 2011, 6, 1269-1282.

A 100-mL round-bottomed flask was charged with L-carnosine (1.41 g, 6.2 mmol), chloroform (CHCI₃, 40 mL), lanosterol (3.2 g, 7.5 mmol) and p-toluenesulfonic acid (3.5 g, 18.8 mmol). The suspension was heated at reflux, and H₂O, which is formed during the reaction, was removed by continuous azeotropic distillation. The distillation temperature was progressively increased and the starting suspension became a solution. After 4 h, the reaction was complete. The solvent was evaporated in vacuo, and the residue was dissolved in MeOH and passed through an ion exchange column to remove p-toluenesulfonic acid. The collected solution was evaporated in vacuo to yield L-carnosine lanosteryl ester as a white solid.

### EXAMPLE 14

alpha Lipoic Acid (ALA) ester of Lanosterol

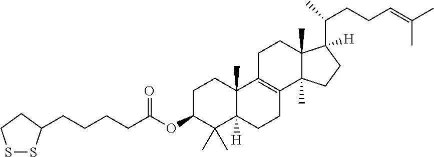 [SMILES: CC(C)=CCC[C@@H](C)[C@H]1CC[C@@]2(C)C3=C(CC[C@]12C)[C@@]1(C)CC[C@H](OC(=O)CCCCC2CCSS2)C(C)(C)[C@@H]1CC3]

Lanosteryl lipoate

Synthetic procedure reported for 1-(cyclohexylmethyl)piperidin-4-ol lipoate: 0. Prezzavento, E. Arena, C. Parenti, L. Pasquinucci, G. Aricò, G. M. Scoto, S. Grancara, A. Toninello, S. Ronsisvalle.

Design and Synthesis of New Bifunctional Sigma [symbol] 1 Selective Ligands with Antioxidant Activity. J. Med. Chem. 2013, 56, 2447-2455.

N,N′-Dicyclohexylcarbodiimide (DCC, 0.45 g, 2.20 mmol) is added to a solution of lanosterol (0.94 g, 2.20 mmol), lipoic acid (0.45 g, 2.20 mmol), and N,N-dimethylamino)pyridine (DMAP, 0.22 mmol, 0.026 mg) in dry dichloromethane (DCM, 5.4 mL) under stirring and nitrogen atmosphere at 0° C. After 10 min, the reaction temperature is slowly raised to room temperature and the reaction mixture is stirred for over 3 h. The dicyclohexylurea (DCU) that precipitated is removed by filtration through a fritted Büchner funnel. The filtrate is washed twice with sodium bicarbonate solution (5%, 10 mL) and twice with brine solution (10 mL). After drying (Na₂SO₄), the solvent is removed under reduced pressure. During this procedure, the additional precipitated DCU is removed by several filtrations. The organic phase is concentrated and purified by flash chromatography (silica) using ethyl acetate/cyclohexane to yield lanosteryl lipoate.

### EXAMPLE 15

Phosphoric acid monoester of Lanosterol

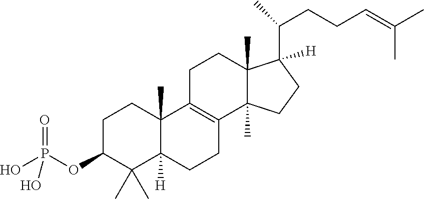 [SMILES: CC(C)=CCC[C@@H](C)[C@H]1CC[C@@]2(C)C3=C(CC[C@]12C)[C@@]1(C)CC[C@H](OP(=O)(O)O)C(C)(C)[C@@H]1CC3]

Phosphoric acid monoester of lanosterol

R. J. W. Cremlyn, N. A. Olsson. Some Steroid Phosphates and Related Compounds. J. Chem. Soc. (C), 1969, 2305-2310.

Lanosterol (5 g) in pyridine (25 mL) was added dropwise during 4 h to a stirred solution of phosphorus oxychloride (5 mL) in acetone (25 mL) at 0° C., to give lanosteryl phosphorodichloridate as a cream powder (3.1 g, 49%), m.p. 113° C. (decomp.) (Found: C, 66.1 ; H, 9.0; CI 13.0; P, 5.5. C₃₀H₄₉Cl₂O₂P requires C, 66.3; H, 9.0; Cl, 12.9; P, 5.7%). νₘₐₓ 1300 (P═O), 1010 (P—O—C) cm⁻¹.

Lanosteryl phosphorodichloridate (500 mg) was boiled under reflux with dioxan (10 mL)-water (1 mL) for 4 h. The solution was cooled to give lanosteryl phosphate as platelets (380 mg, 78%), mp 204-205° C. (Found: C, 70.4; H, 10.0; P, 6.5. C₃₀H₅₁O₄P requires C, 70.7; H, 10.6; P, 6.1%).

### EXAMPLE 16

Glycine ester of Lanosterol

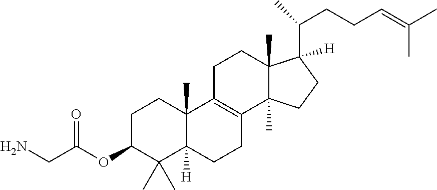 [SMILES: CC(C)=CCC[C@@H](C)[C@H]1CC[C@@]2(C)C3=C(CC[C@]12C)[C@@]1(C)CC[C@H](OC(=O)CN)C(C)(C)[C@@H]1CC3]

Glycine lanosteryl ester

Synthetic procedure reported for cholesterol glycine ester:

Arghajit Pyne, Jagannath Kuchlyan, Chiranjit Maiti, Dibakar Dhara, Nilmoni Sarkar. Cholesterol Based Surface Active Ionic Liquid That Can Form Microemulsions and Spontaneous Vesicles.

Langmuir 2017, 33, 5891-5899.

N-(tert-Butyloxycarbonyl) glycine (Boc-Gly, 1.5 g, 8.56 mmol), lanosterol (3.64 g, 8.54 mmol), and N,N-dimethylamino)pyridine (DMAP, 0.1 g, 0.82 mmol) are dissolved in a 100-mL double necked round-bottomed flask containing a magnetic stir bar using dry dichloromethane (DCM, 40 mL). The flask is kept in an ice-water bath with maintaining nitrogen atmosphere in entire reaction medium. To this solution, N,N′-dicyclohexylcarbodiimide (DCC, 1.76 g, 8.54 mmol) in DCM (10 mL) is added in a drop wise manner over 30 min with vigorous stirring. The reaction mixture is kept for an additional 30 min in an ice-water bath and then left to stir for overnight at room temperature. The resulting insoluble N,N′-dicyclohexylurea is filtered off and the filtrate is washed with 1 M HCI and dried over anhydrous MgSO₄. Finally, the product is purified by column chromatography on silica using 10% ethyl acetate in hexane as the eluent to yield white solid. In the next step, Boc group deprotection is performed using trifluoroacetic acid (TFA) in dry DCM. For this 1.5 g (2.57 mmol) of the lanosteryl-Boc-Gly is dissolved in dry DCM in (10 mL) a 25-mL double necked round-bottomed flask, and 0.25 mL TFA (3.27 mmol) in dry DCM (3 mL) is added dropwise. The solution is kept at room temperature under a nitrogen atmosphere with continuous stirring for 24 h. After that TFA is removed by work up with saturated NaHCO₃ solution, and finally the product is purified by column chromatography using methanol/ethyl acetate (1:1) as the eluent to yield white solid.

The advantage presented by glycine ester of lanosterol is its increased aqueous solubility compared to parent compound lanosterol.

### EXAMPLE 17

Sulfuric acid derivative of Lanosterol

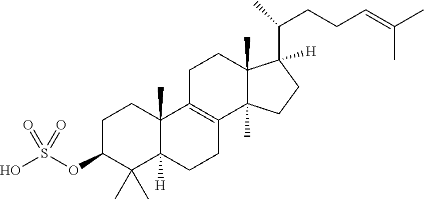 [SMILES: CC(C)=CCC[C@@H](C)[C@H]1CC[C@@]2(C)C3=C(CC[C@]12C)[C@@]1(C)CC[C@H](OS(=O)(=O)O)C(C)(C)[C@@H]1CC3]

sulfuric acid monoester of lanosterol

Synthetic procedure reported for 25-hydroxycholesteryl 3-sulfate (see below):

Shoujiro Ogawa, Genta Kakiyama, Akina Muto, Atsuko Hosoda, Kuniko Mitamura, Shigeolkegawa, Alan F. Hofmann, Takashilida lida

A facile synthesis of C-24 and C-25 oxysterols by in situ generated ethyl(trifluoromethyl)dioxirane

Steroids 2009, 74, Issue 1, Pages 81-87

This procedure was based on earlier work

Dusza J P, Joseph J P, Bernstein S.

The preparation of estradio-17β sulfates with triethylamine-sulfur trioxide.

Steroids 1985, 45, 303-15.

Sulfur trioxide-trimethylamine complex (30 mg, 0.22 mmol) is added to a solution of lanosterol (30 mg, 0.07 mmol) in dry pyridine (2 mL), and the suspension is stirred at room temperature for 1 h. The reaction mixture is poured onto ice-cooled petroleum ether (20 mL) and the precipitated solid is collected by filtration. After being washed with petroleum ether, the solid product is dissolved in methanol (1 mL). The resulting solution is adjusted to pH 8 by adding 1 M NaOH, diluted with water (10 mL), and then loaded onto a preconditioned Sep-Pak Vac tC₁₈ cartridge. The cartridge is successively washed with water (20 mL) and then with 20% methanol (20 mL), and the desired lanosteryl 3-sulfate is eluted with methanol (20 mL). After evaporation of the solvent, recrystallization of the residue from methanol—EtOAc gives the sodium salt of lanosteryl 3-sulfate as a colorless solid.

The advantage presented by lanosteryl sulfate is its increased aqueous solubility compared to parent compound lanosterol.

### EXAMPLE 18

tert-pentanoic acid ester of Lanosterol

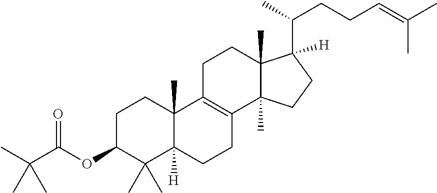 [SMILES: CC(C)=CCC[C@@H](C)[C@H]1CC[C@@]2(C)C3=C(CC[C@]12C)[C@@]1(C)CC[C@H](OC(=O)C(C)(C)C)C(C)(C)[C@@H]1CC3]

Lanosteryl pivalate

Synthetic procedure reported for cholesterol 3β-pivalate: Schwarz, V.; Hermanek, S.; Trojanek, J.

Steroid derivatives. Xl. The effect of 3β-substituents on the rate of bromine addition to derivatives of 5-cholestene.

Collection of Czechoslovak Chemical Communications 1961, 26, 1438-1442.

A solution of 1.5 g lanosterol in 6 mL pyridine is treated with 1.3 mL pivaloyl chloride. The reaction mixture is allowed to stand at room temperature overnight. Working up the reaction in a conventional way gives colorless needles of the desired product.

### EXAMPLE 19

NAC ester of 25-Hydroxycholesterol

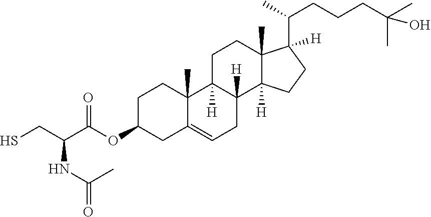

3-(N-Acetyl-L-cysteine) ester of 25-hydroxycholesteryl

Note that the secondary hydroxyl group on the A ring in 25-hydroxycholesterol is more reactive than the tertiary alcohol in the side chain so protection of the 25-hydroxyl group is not necessary to get selective reaction at position 3.

Synthetic procedure reported for the preparation of amides of N-acetyl-L-cysteine: Uzma I. Zakai, Galina Bikzhanova, Daryl Staveness, Stephen Gately, Robert West. Synthesis of lipophilic sila derivatives of N-acetylcysteineamide, a cell permeating thiol.

Appl. Organometal. Chem. 2010, 24, 189-192.

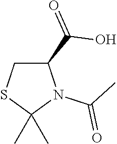

(R)-4-carboxy-3-acetyl-2,2-dimethylthiazolidine was prepared as follows. A suspension of N-acetyl-L-cysteine (1.0 g, 6 mmol) and montmorillonite K10 (0.2 g, 20 wt %) in anhydrous acetone-2,2-dimethoxypropane mixture (1:3, 40 mL) was stirred at room temperature for 3 h. The reaction mixture was then filtered, and solvent was evaporated to give (R)-4-carboxy-3-acetyl-2,2-dimethylthiazolidine (1.12 g, 95% yield) as white solid (90% pure) which was further purified by recrystallization from acetone-hexane (1.03 g, 84% yield). ¹H NMR (acetone-d₆, 500 MHz) δ 1.48 (s, 3 H, Me), 1.50 (s, 3 H, Me), 1.95 (s, 3 H, NAc), 2.86 (dd, J=13.3, 7.1, 1 H, CHH), 3.00 (dd, J=13.3, 5.1, 1 H, CHH), 4.67 (ddd, J=8.0, 7.1, 5.1, 1 H, CH), 12.80 (br. s, 1 H, OH).

A solution of (R)-4-carboxy-3-acetyl-2,2-dimethylthiazolidine (1 mmol) and triethylamine (1 mmol) in dichloromethane (DCM, 4 mL) is cooled to −5° C., and a solution of ethyl chloroformate (1 mmol) in dichloromethane (1 mL) is added dropwise. After 15 min of stirring at −5° C., 25-hydroxycholesterol (1 mmol) is slowly added to the reaction mixture. Stirring is continued for 25 min at −5° C. and 15 h at room temperature.

The reaction mixture is then diluted with DCM (6 mL) and washed thoroughly with portions of 5% hydrochloric acid, sodium bicarbonate and water (6 mL each). The dichloromethane solution is dried (MgSO₄) and evaporated to give the dimethylthiazolidine ester intermediate. A solution of the dimethylthiazolidine ester intermediate in 2 M HCI methanolic solution (15 mL) is stirred at room temperature for 24 h. The methanol is removed under reduced pressure and the reaction worked up with DCM-brine. The dichloromethane solution is dried (MgSO₄), filtered and evaporated to give the 25-hydroxycholesterol ester of N-acetyl-L-cysteine.

### EXAMPLE 20

N-Acetylcarnosine ester of 25-hydroxycholesteryl

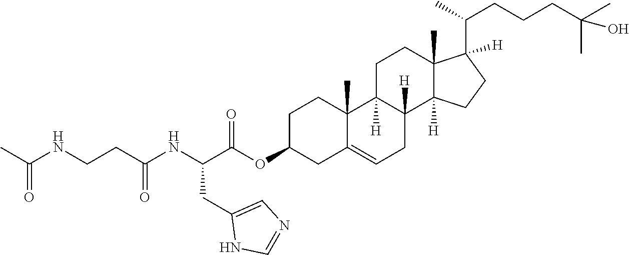

3-(N-Acetyl-L-carnosine) 25-hydroxycholesteryl ester

Note that the secondary hydroxyl group on the A ring in 25-hydroxycholesterol is more reactive than the tertiary alcohol in the side chain so protection of the 25-hydroxyl group is not necessary to get selective reaction at position 3.

Synthetic procedure reported for the esterification of N-acetylamino acids: Thonthula Sreelatha, Atmakur Hyavathi, Katragadda Suresh Babu, Joish Madhusudana Murthy, Usha Rani Pathipati, Janaswamy Madhusudana Rao. Synthesis and Insect Antifeedant Activity of Plumbagin Derivatives with the Amino Acid Moiety.

J. Agric. Food Chem. 2009, 57, 6090-6094.

N,N′-Dicyclohexylcarbodimide (DCC, 1.5 mmol) and a catalytic amount of 3-hydroxybenzotriazole are added to a cooled solution (0° C.) of 25-hydroxycholesterol (1 mmol) in dry dichloromethane (DCM, 10 mL) under a nitrogen atmosphere. After stirring for 15 min, N-acetyl-L-carnosine (1.2 mmol) is added, and stirring is continued at room temperature. After completion of the reaction (monitored by TLC), the reaction mixture is filtered to remove the precipitated dicyclohexylurea. The filtrate is evaporated under reduced pressure, and the residue is purified by silica gel (100-200 mesh) column chromatography using dichloromethane/methanol as eluent to afford the lanosteryl ester of N-acetyl-L-carnosine.

### EXAMPLE 21

Lipoate Ester of 25-hydroxycholesteryl

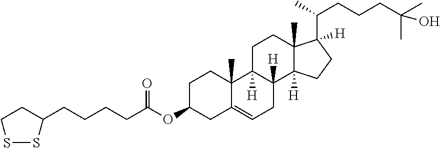

25-Hydroxycholesteryl 3-lipoate

Note that the secondary hydroxyl group on the A ring in 25-hydroxycholesterol is more reactive than the tertiary alcohol in the side chain so protection of the 25-hydroxyl group is not necessary to get selective reaction at position 3.

Synthetic procedure reported for 1-(cyclohexylmethyl)piperidin-4-ol lipoate:

0. Prezzavento, E. Arena, C. Parenti, L. Pasquinucci, G. Aricò, G. M. Scoto, S. Grancara, A. Toninello, S. Ronsisvalle.

Design and Synthesis of New Bifunctional Sigma [symbol] 1Selective Ligands with Antioxidant Activity.

J. Med. Chem. 2013, 56, 2447-2455.

N,N′-Dicyclohexylcarbodiimide (DCC, 0.45 g, 2.20 mmol) is added to a solution of 25-hydroxycholesterol (0.94 g, 2.20 mmol), lipoic acid (0.45 g, 2.20 mmol), and N,N-dimethylamino)pyridine (DMAP, 0.22 mmol, 0.026 mg) in dry dichloromethane (DCM, 5.4 mL) under stirring and nitrogen atmosphere at 0° C. After 10 min, the reaction temperature is slowly raised to room temperature and the reaction mixture is stirred for over 3 h. The dicyclohexylurea (DCU) that precipitated is removed by filtration through a fritted Büchner funnel. The filtrate is washed twice with sodium bicarbonate solution (5%, 10 mL) and twice with brine solution (10 mL). After drying (Na₂SO₄), the solvent is removed under reduced pressure. During this procedure, the additional precipitated DCU is removed by several filtrations. The organic phase is concentrated and purified by flash chromatography (silica) using ethyl acetate/cyclohexane.

### EXAMPLE 22

Sulfate derivative of 25-Hydroxycholesterol

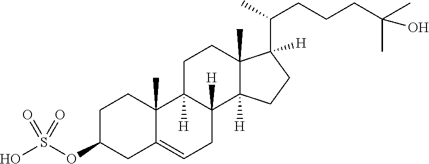

25-Hydroxycholesteryl 3-sulfate (3β-Sulfooxy-25-hydroxycholest-5-ene)

Shoujiro Ogawa, Genta Kakiyama, Akina Muto, Atsuko Hosoda, Kuniko Mitamura, Shigeolkegawa, Alan F. Hofmann, Takashilida lida

A facile synthesis of C-24 and C-25 oxysterols by in situ generated ethyl(trifluoromethyl)dioxirane

Steroids 2009, 74, Issue 1, Pages 81-87

This procedure was based on earlier work

Dusza JP, Joseph JP, Bernstein S.

The preparation of estradio17β sulfates with triethylamine-sulfur trioxide.

Steroids 1985, 45, 303-15.

Sulfur trioxide-trimethylamine complex (30 mg, 0.22 mmol) is added to a solution of 25-hydroxycholesterol (30 mg, 0.07 mmol) in dry pyridine (2 mL), and the suspension is stirred at room temperature for 1 h. The reaction mixture is poured onto ice-cooled petroleum ether (20 mL) and the precipitated solid is collected by filtration. After being washed with petroleum ether, the solid product is dissolved in methanol (1 mL). The resulting solution is adjusted to pH 8 by adding 1 M NaOH, diluted with water (10 mL), and then loaded onto a preconditioned Sep-Pak Vac tC₁₈ cartridge. The cartridge is successively washed with water (20 mL) and then with 20% methanol (20 mL), and the desired 25-hydroxychloesteryl 3-sulfate is eluted with methanol (20 mL). After evaporation of the solvent, recrystallization of the residue from methanol—EtOAc gives the analytically pure product in the form of a colorless amorphous solid: yield, 25 mg (70%). mp 164-165° C. IR (KBr) νₘₐₓ cm⁻¹: 3451 (OH). ¹H NMR (CD₃OD) δ: 0.72 (3H, s, 18-CH₃), 0.96 (3H, d, J 5.4, 21-CH₃), 1.03 (3H, s, 19-CH₃), 1.17 (6H, s, 26- and 27-CH3), 4.13 (1H, br. m, 3α-H), 5.38 (1H, br. s, 6-H). LR-MS (FAB⁻), m/z: 481 (M⁻, 71%), 306 (10%), 199 (12%), 168 (15%), 153 (100%), 122 (22%), 97 (HSO₄⁻68%), 80 (SO₃⁻, 38%). HR-MS (FAB⁻), calculated for C₂₇H₄₅O₅S, 481.2987; found m/z: 481.2992.

### EXAMPLE 23

Glutathione ester of Lanosterol

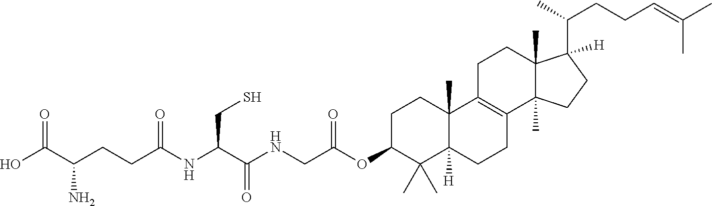 [SMILES: CC(C)=CCC[C@@H](C)[C@H]1CC[C@@]2(C)C3=C(CC[C@]12C)[C@@]1(C)CC[C@H](OC(=O)CNC(=O)[C@H](CS)NC(=O)CC[C@H](N)C(=O)O)C(C)(C)[C@@H]1CC3]

Glutathione lanosteryl ester

Synthetic procedure reported for glutathione ester of carotenoids:

Lockwood, Samuel Fournier; O'Malley, Sean; Watumull, David G.; Hix, Laura M.; Jackson, Henry; Nadolski, Geoff.

Preparation of carotenoid ester analogs or derivatives for the inhibition and amelioration of ischemic reperfusion injury.

Patent Information: Jan. 08, 2008, US 7317008, B2

Assignee: Cardax Pharmaceuticals, Inc., USA

Diisopropylethylamine (DIPEA, 0.878 mL, 5.04 mmol), 1-hydroxybenzotriazole hydrate (HOBT-H₂O, 0.3094 g, 2.02 mmol), 4-(dimethylamino)pyridine (DMAP, 0.4105 g, 3.36 mmol), N,N′-diisopropylcarbodiimide (DIC, 0.316 mL, 2.02 mmol), and lanosterol (0.0717 g, 0.168 mmol) were added to a suspension of reduced glutathione (0.5163 g; 1.68 mmol) in dichloromethane (DCM, 3 mL)/dimethylformamide (DMF, 3 mL) at room temperature. The reaction mixture was stirred at room temperature for 36 h; at which time the reaction was diluted with DCM, quenched with brine/1 M HCI (20 mL/3 mL), and then extracted with DCM. The combined organic layers were concentrated to yield glutathione monoester.

Thus, while there have been shown, described and pointed out, fundamental novel features of the present disclosure as applied to the exemplary embodiments thereof, it will be understood that various omissions and substitutions and changes in the form and details of devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

1-42. (canceled)

43. A compound of the formula 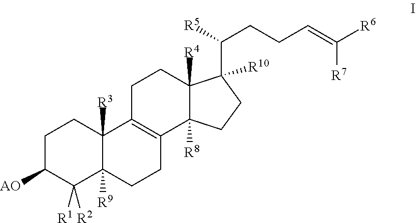
or a pharmaceutically acceptable salt thereof, wherein R¹ through R⁸ are each independently selected from hydrogen or lower alkyl optionally substituted by one to three fluoro;
R⁹ and R¹° are each independently selected from hydrogen and fluoro; and
A is an esterified hydroxyl derivative to formula I at A, the hydroxyl derivative including N-acetylcysteine (NAC), alpha-lipoic acid, N-acetylcarnosine, glycine, pivalic acid, arginine and glutathione.

44. The compound according to claim 43, wherein the compound is of the formula 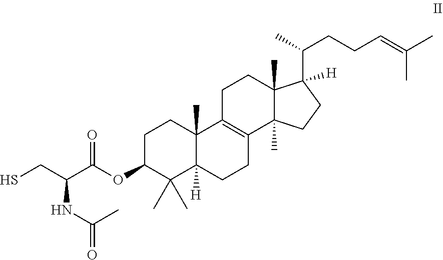 [SMILES: CC(=O)N[C@@H](CS)C(=O)O[C@H]1CC[C@]2(C)C3=C(CC[C@H]2C1(C)C)[C@]1(C)CC[C@H]([C@H](C)CCC=C(C)C)[C@@]1(C)CC3]
or a pharmaceutically acceptable salt thereof.

45. The compound according to claim 43, wherein the compound is of the formula 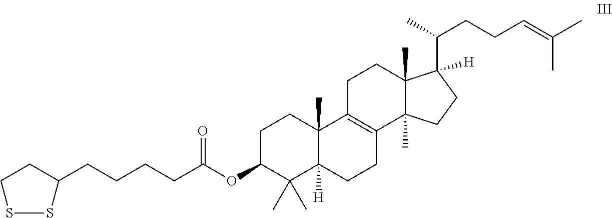 [SMILES: CC(C)=CCC[C@@H](C)[C@H]1CC[C@@]2(C)C3=C(CC[C@]12C)[C@@]1(C)CC[C@H](OC(=O)CCCCC2CCSS2)C(C)(C)[C@@H]1CC3]
or a pharmaceutically acceptable salt thereof.

46. The compound according to claim 43, wherein the compound is of the formula 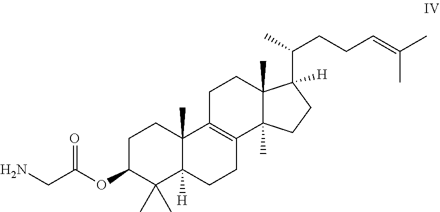 [SMILES: CC(C)=CCC[C@@H](C)[C@H]1CC[C@@]2(C)C3=C(CC[C@]12C)[C@@]1(C)CC[C@H](OC(=O)CN)C(C)(C)[C@@H]1CC3]
or a pharmaceutically acceptable salt thereof.

47. A method of treating an eye cataract in a mammal in need of such treatment comprising administering to said mammal a therapeutically effective amount of a compound of claim 43.

48. A method of treating an eye presbyopia in a mammal in need of such treatment comprising administering to said mammal a therapeutically effective amount of a compound of claims 43.

49. A compound of the formula 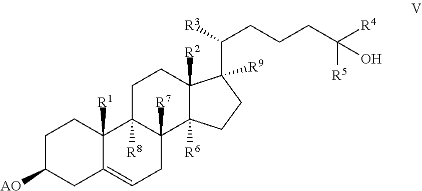
or a pharmaceutically acceptable salt thereof, R¹ through R⁸ are each independently selected from hydrogen or lower alkyl optionally substituted by one to three fluoro;
R⁹ is selected from hydrogen and fluoro; and
A is a hydroxyl derivative esterified to formula V at A, the hydroxyl derivative including N-acetylcysteine (NAC), alpha-lipoic acid, N-acetylcarnosine, glycine, pivalic acid, arginine and glutathione.

50. The compound according to claim 49, wherein the compound is of the formula 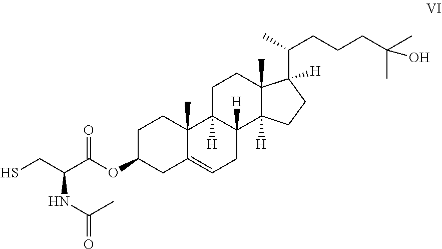
or a pharmaceutically acceptable salt thereof.

51. The compound according to claim 49, wherein the compound is of the formula 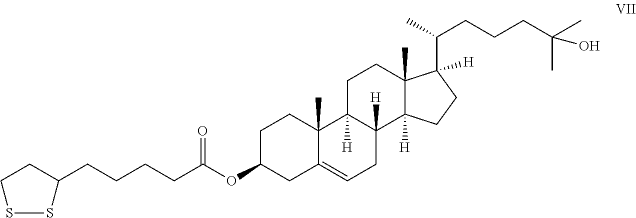
or a pharmaceutically acceptable salt thereof.

52. The compound according to claim 49, wherein the compound is of the formula 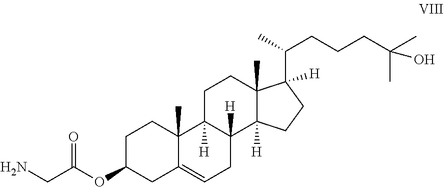
or a pharmaceutically acceptable salt thereof.

53. A method of treating an eye cataract in a mammal in need of such treatment comprising administering to said mammal a therapeutically effective amount of a compound of claim 49.

54. A method of treating an eye presbyopia in a mammal in need of such treatment comprising administering to said mammal a therapeutically effective amount of a compound of claim 49.

55. A method of treating an eye cataract in a mammal in need of such treatment comprising administering an effective amount of lanosterol or a derivative thereof and an oxidative protective agent, wherein the lanosterol derivative is a compound of claim 43 and with lanosterol the oxidative protective agent is N-acetylcarnosine, N-acetylcysteine or N-acetylcysteine amide.

56. A method of treating an eye cataract in a mammal in need of such treatment comprising administering an effective amount of 25-hyroxycholesterol or a derivative thereof and an oxidative protective agent, wherein the 25-hyroxycholesterol derivative is a compound of claim 49 and with 25-hyroxycholesterol, the oxidative protective agent is N-acetylcysteine, N-acetylcarnosine or N-acetylcysteine amide.

57. A method of treating an eye presbyopia in a mammal in need of such treatment comprising administering an effective amount of lanosterol or a derivative thereof and an oxidative protective agent, wherein the lanosterol derivative is a compound of claim 43 and with lanosterol, the oxidative protective agent is N-acetylcarnosine, N-acetylcysteine or N-acetylcysteine amide.

58. A method of treating an eye presbyopia in a mammal in need of such treatment comprising administering an effective amount of 25-hyroxycholesterol or a derivative thereof and an oxidative protective agent, wherein the 25-hyroxycholesterol derivative is a compound of claim 49 and with 25-hyroxycholesterol, the oxidative protective agent is N-acetylcysteine, N-acetylcarnosine or N-acetylcysteine amide.

59. An ophthalmic composition comprising lanosterol or a derivative thereof wherein the lanosterol derivative is a compound of claim 43 and at least one compound selected from N-acetylcysteine (NAC), N-acetylcysteine amide (NACA) and N-acetylcarnosine in a physiologically acceptable buffer, having a pH of 5.0 to 8.0, wherein said lanosterol or a derivative thereof are present at a concentration ranging from about 0.010% w/v to about 5% w/v and each of said compounds N-acetylcysteine (NAC), N-acetylcysteine amide (NACA) or N-acetylcarnosine is present at a concentration ranging from about 0.01% w/v to about 2.00% w/v.

60. An ophthalmic composition comprising 25-hydroxycholesterol or a derivative thereof wherein the 25-hydroxycholesterol derivative is a compound of claim 49 and at least one compound selected from N-acetylcysteine amide (NACA), N-acetylcysteine, and N-acetylcarnosine in a physiologically acceptable buffer, having a pH of 5.0 to 8.0, wherein said 25-hydroxycholesterol or a derivative thereof are present at a concentration ranging from about 0.010% w/v to about 5% w/v and each of said compounds N-acetylcysteine (NAC), N-acetylcysteine amide (NACA) or N-acetylcarnosine is present at a concentration ranging from about 0.01% w/v to about 2.00% w/v.

61. A method of modulating proteins in an eye of a mammal in need of such treatment comprising administering to said mammal an effective amount of a compound of claim 43.

62. A method of modulating proteins in an eye of a mammal in need of such treatment comprising administering to said mammal an effective amount of a compound of claim 49.
